(19) United States
(12) Patent Application Publication
(10) Pub. No.: US 2021/0115506 A1
(43) Pub. Date: Apr. 22, 2021

(54) Quantification of transplant-derived circulating cell-free DNA in the absence of a donor genotype

(71) Applicant: The Board of Trustees of the Leland Stanford Junior University, Stanford, CA (US)

(72) Inventors: Iwijn De Vlaminck, Ithaca, NY (US); Eilon Sharon, Menlo Park, CA (US); Jonathan Pritchard, Stanford, CA (US); Stephen R Quake, Stanford, CA (US); Hannah Valantine, Bethesda, MD (US); Kiran Khush, Stanford, CA (US)

(21) Appl. No.: 16/500,533

(22) PCT Filed: Apr. 2, 2018
(86) PCT No.: PCT/US2018/025719

§ 371 Date: Oct. 3, 2019

Related U.S. Application Data
(60) Provisional application No. 62/481,262, filed on Apr. 4, 2017.

(51) Int. Cl. C12Q 1/6869; G16B 20/00

(52) U.S. Cl. CPC C12Q 1/6869; C12Q 2545/114; G16B 20/00

(57) ABSTRACT

Prediction of allograft rejection is provided based on the quantification of transplant-derived circulating cell-free DNA (dd-cfDNA levels) in the absence of a donor genotype. The technology provided herein alleviates some of the barriers to the implementation of Genome Transplant Dynamics (GTD), which will further widen its clinical application.

### FIELD OF THE INVENTION

This invention relates to methods and systems for measuring allograft or host injury. Specifically, the invention relates to methods and system of donor-derived (dd-cfDNA) monitoring using shotgun sequencing without donor genotype information.

### BACKGROUND OF THE INVENTION

Although solid-organ transplantation is now a common practice, the clinical outcomes remain poor with median survival rate (5.3 years for lung and 11 years for heart). Accurate monitoring of allograft health is essential for long-term survival of the transplant recipient. The current gold standard method of allograft rejection surveillance is the biopsy (transbronchial biopsy for lung transplant and endomyocardial biopsy for heart transplant), but this invasive technique suffers from high cost and myriad complications.

Current implementation of shotgun-based Genome Transplant Dynamics (GTD), which we will refer to as the “two-genomes” method, requires genotyping of both the donor and recipient. In contrast to the recipient genotype that is easy to obtain, the donor genotype is often unavailable. We therefore in this invention set out to develop a method that enables dd-cfDNA monitoring using shotgun sequencing without donor genotype information—a “one-genome” method. In this invention as exemplary embodiments, we apply the method to lung and heart transplant recipient cohort data and demonstrate that the performance of a one-genome method approaches the performance of the two-genome method.

### SUMMARY OF THE INVENTION

Quantification of cell-free DNA (cfDNA) in circulating blood derived from a transplanted organ is a powerful approach to monitoring post-transplant injury. Genome Transplant Dynamics (GTD) quantifies donor-derived cfDNA (dd-cfDNA) by taking advantage of single-nucleotide polymorphisms (SNPs) distributed across the genome to discriminate donor and recipient DNA molecules. In its current implementation, GTD requires genotyping of both the transplant recipient and donor. However, in practice, donor genotype information is often unavailable.

In this invention, we address this issue by developing an algorithm that estimates dd-cfDNA levels in the absence of a donor genotype. The method predicts heart and lung allograft rejection with an accuracy that is similar to conventional GTD. We furthermore refined the method to handle closely related recipients and donors, a scenario that is common in bone marrow and kidney transplantation. We show that it is possible to estimate dd-cfDNA in bone marrow transplant patients that are unrelated or that are siblings of the donors, using a hidden Markov model (HMM) of identity-by-descent (IBD) states along the genome. Last, we demonstrate that comparing dd-cfDNA to the proportion of donor DNA in white blood cells can differentiate between relapse and the onset of graft-versus-host disease (GVHD). These methods alleviate some of the barriers to the implementation of GTD, which will further widen its clinical application.

In an exemplary embodiment, the present invention provides a method of quantifying transplant-derived circulating donor-derived cell-free DNA in the absence of a donor genotype according to the following steps: (a) extracting a blood sample from a transplant recipient; (b)isolating circulating nucleotide acids from the plasma of the extracted blood sample; (c) performing an unbiased sequencing on the isolated circulating nucleotide acids using a first computer-implemented method; (d) estimating and quantifying the donor-derived cell-free DNA based on genotyping from only the transplant recipient using a second computer-implemented method; and (e) outputting the quantified transplant-derived circulating donor-derived cell-free DNA. Both computer-implemented methods and the outputting are executed by a computer device. It is noted that the method does not use targeted PCR amplification. The step of genotyping from only the transplant recipient could be based on genotyping array, cell-free DNA sequencing or low coverage whole genome sequencing followed by imputations.

Transplant recipients are solid-organ transplant recipients (e.g. heart, lung, kidney, liver, or the like), bone marrow transplant recipients or hematopoietic stem cell recipients.

Embodiments of this invention could be implemented as a method, computer-implemented method, software or system. Some method steps could be implemented as computer-implemented steps executable by computer hardware, device, chip, system and/or processor.

### BRIEF DESCRIPTION OF THE DRAWINGS

FIG. 1 shows according to an exemplary embodiment of the method of the invention. (a) Graphical illustration of the “one-genome” statistical model for dd-cfDNA estimation in unrelated individuals. Parameters (relatively lighter gray background), hidden parameters and data (relatively draker gray background) are represented by text boxes. (b) when the individuals may be closely related (in this invention, in case of a bone marrow transplant) the donor genotype depends on the recipient genotype and the identity by descent (IBD) state between the recipient and donor genptypes. IBD states are modeled for blocks of ˜2 cM along the genome. Transition between IBD states depends on the number of meioses that separate each pair of recipient-donor chromosomes given their most recent diploid common ancestor (MRCA 1 and MRCA 2).

FIGS. 2A-D show according to an exemplary embodiment of the invention comparison of predicted levels of dd-cfDNA by one- and two-genomes methods in heart and lung transplant recipients. FIG. 2A and FIG. 2B comparison between levels of dd-cfDNA predicted by the two-genomes method (x-axis) and the one-genome method (y-axis). FIG. 2C and FIG. 2D show a comparison of one- and two-genomes methods predictability of organ rejection. Each bar shows the area under the curve (AUC) of discriminating between two rejection states as measured using biopsies using dd-cfDNA fraction estimates. Error bars marks AUC 95% confidence interval. The significance of the difference between corresponding receiver operating characteristic (ROC) of the one-genome and two-genomes was done using DeLong two-sided test. The gray levels of the one- and two-genomes bars appear the same. The reader should interpret each gray block of bars, the bottom bar to be one-genome and the top bar to be the two-genome.

FIGS. 3A-B show according to an exemplary embodiment of the invention a comparison of predicted levels of dd-cfDNA by one- and two-genomes methods in bone marrow transplant recipients. FIG. 3A, comparison between levels of dd-cfDNA predicted by the two-genomes method (x-axis) and the one-genome method (y-axis) when learning donor and recipient relations (orange) or naively assuming that they are unrelated (blue). The later underestimates dd-cfDNA levels when the recipient and donor are siblings. Dashed lines show 1:1 and 2:1 ratios. FIG. 3B, an example of cfDNA level estimates in a single bone marrow transplant recipient that is a sibling of the donor (16).

FIG. 4 shows according to an exemplary embodiment of the invention comparing the fraction of cfDNA that is recipient-derived to the fraction of recipient-derived blood cells may detect GVHD. A proof of principle in a single bone marrow transplant recipient (patient I8), that differences in the recipient-derived cfDNA and recipient-derived blood cells levels may indicate onset of GVHD. The difference between the two measurements is due to injured tissue-derived cfDNA. In contrast, when relapse occurs both measurements should show an increase in recipient-derived fraction (not shown in this figure). This may help to distinguish between GVHD and relapse in bone-marrow transplanted patients.

FIG. 5 shows according to an exemplary embodiment of the invention cfDNA sequencing and genotyping data processing pipeline. Illustration of the pipeline used to retrieve allele counts in cfDNA fragments for each recipient-genotyped SNP from the raw cfDNA sequencing and genotyping measurements.

FIGS. 6A-D show according to an exemplary embodiment of the invention for different patients a comparison between predicted levels of dd-cfDNA and the fraction of reads that map to the X chromosome when recipient and donor sex are different. Patients I1, I2 and I4 the recipient are males with female donors; patient I8 is a female with a male donor.

FIG. 7 shows according to an exemplary embodiment of the invention. A comparison between one- and two-genomes methods predictions of cfDNA levels in bone marrow transplant patients. Each panel shows results for a single patient. Dotted-dash line marks day in which engraftment was detected (absolute neutrophil count (ANC)>500 for three consecutive days). Purple dashed lines mark clinical diagnoses. It can be seen that the predictions of one-genome method that learns IBD are similar to the prediction of the two-genomes method, while fixing the one-genome method to non-related recipient and donor state (IBD=0) underestimate the dd-cfDNA fraction.

FIGS. 8A-B show according to an exemplary embodiment of the invention a comparison of prediction diagnosis using estimation of dd-cfDNA levels by one- and two-genomes methods in heart and lung transplant recipients. FIG. 8A and FIG. 8B show a comparison of one- and two-genomes methods predictability of organ rejection. In opposed to FIGS. 2A-D, here the two-genome prediction were not corrected by Error estimation. Each bar shows the area under the curve (AUC) of discriminating between two rejection states as measured using biopsies using dd-cfDNA fraction estimates. Error bars marks AUC 95% confidence interval. The significance of the difference between corresponding receiver operating characteristic (ROC) of the one-genome and two-genomes was done using DeLong two sided test [31,32]. The gray levels of the one- and two-genomes bars appear the same. The reader should interpret each gray block of bars, the bottom bar to be one-genome and the top bar to be the two-genome.

FIGS. 9A-C show according to an exemplary embodiment of the invention show the absolute difference as function of the two-genomes prediction for respectively lung, heart, and bone marrow recipients in FIG. 9A, FIG. 9B and FIG. 9C.

### DETAILED DESCRIPTION

### Quantifying Dd-cfDNA in Lung and Heart Transplant Recipients

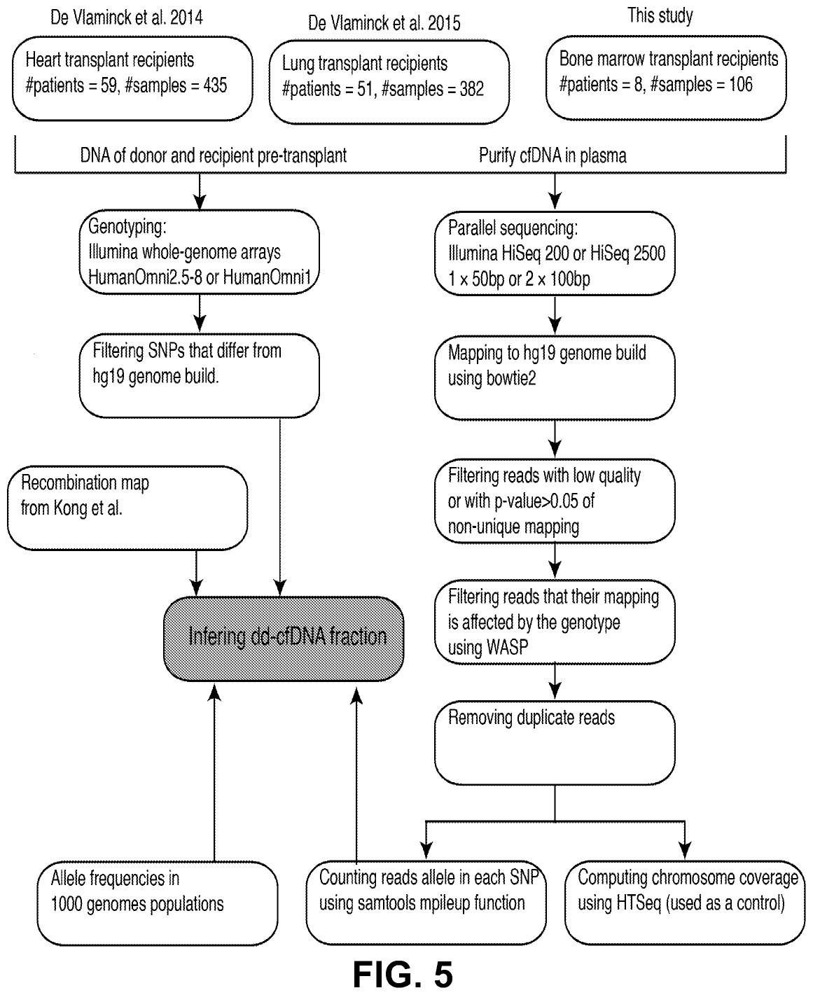

We developed a statistical approach and computer-software method that quantifies donor- and recipient-derived cfDNA fragments in the absence of donor genotype information (see Methods Section infra for a formal description of the model). To quantify the observed abundance of alleles of each genotyped SNP in cfDNA sequences (FIG. 5), we first filter low quality reads, reads that are not mapped uniquely to the genome, and reads with potential for mapping biased by genetic variability. We then remove duplicated reads and count allele appearances of each genotyped SNP (SAMtools mpileup function). We use all genotyped SNPs, as opposed to the “two genomes” method that uses only SNPs that are homozygous but differ between recipient and donor. The observed allele appearances in cfDNA and the recipient genotype are the inputs for our “one-genome” model.

To calculate the probability of the observed cfDNA, we first calculate the probability of each possible donor and recipient genotype. Recipient genotype depends on the recipient measured genotype and the genotyping error rate. Vital organ transplants are rarely closely related. Therefore, for heart and lung transplants, our model assumes that the donor genotype is randomly selected from a human population. Given this assumption, the probability of a specific donor allele is its frequency in the population. Our algorithm iterates over 1000 Genomes Project populations and super-populations to detect the most likely ancestral population of the donor. This model achieves satisfying performance in lung and heart transplant but might need refinement for handling bone marrow transplant in which donor and recipients are often related.

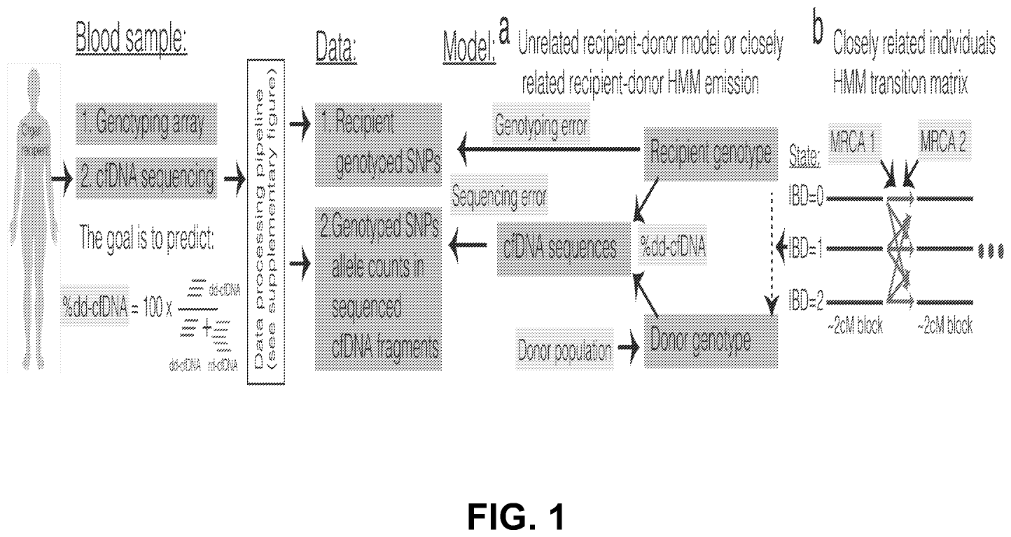

Putting it together, the probability of observing a specific allele in a cfDNA fragment is computed by integrating over all possible recipient and donor genotypes and depends on the sequencing error rate, the fraction of dd-cfDNA in the recipient plasma and the probabilities of observing the allele conditioning on it being donor- or recipient-derived (FIG. 1A indicated (a)). Finally, we compute the log-likelihood of the data by summing log-likelihoods over all SNPs, assuming SNPs are independent (this assumption is also made by the two-genomes method). We use an optimization algorithm to find the maximum likelihood parameter values.

### Performance of Lung and Heart Rejection Predictions

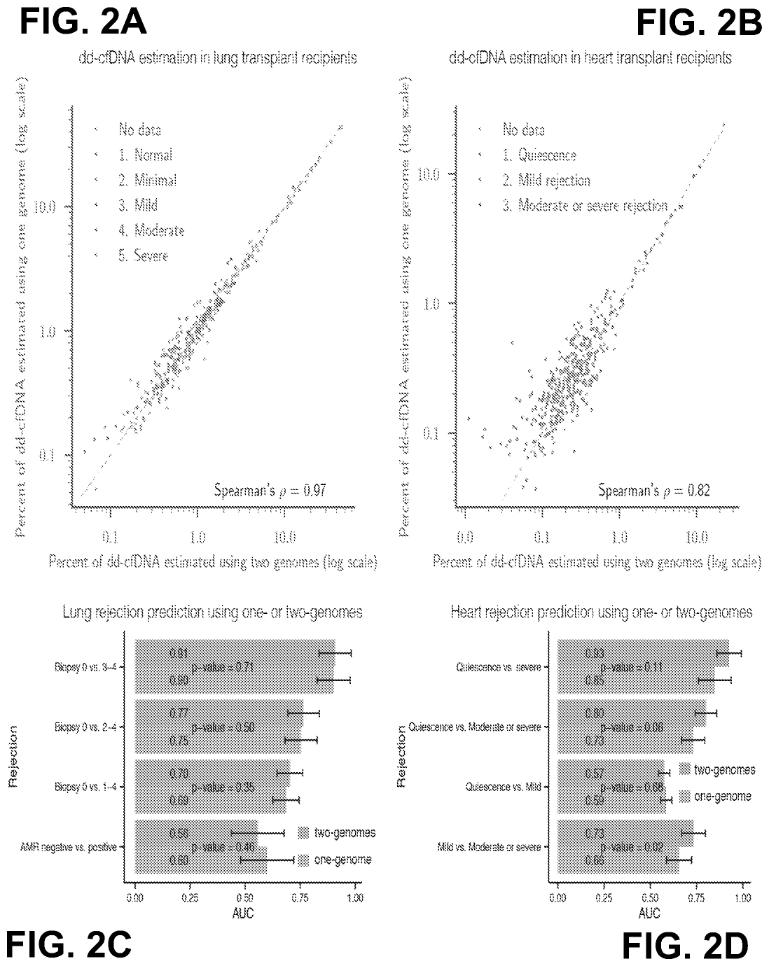

To assess the performance of the one-genome model, we compare estimates of dd-cfDNA for the one and two-genome methods. We find that the predictions of dd-cfDNA level in lung recipients (51 patients, 382 samples) are highly correlated between the two methods (Pearson's R²=0.99, Spearman's ρ=0.94; FIG. 2A). Accordingly, the two methods also performed similarly in differentiating between different levels of organ rejection (as measured by biopsy; FIG. 2C). For heart transplant recipients (59 patients, 435 samples) dd-cfDNA level estimates of the two methods were highly correlated (Pearson's R²=0.99, Spearman's ρ=0.67; FIG. 2B). However, the agreement between the two methods is not as high as in the lung cohort, especially when dd-cfDNA levels are below ˜0.5%. This may be because samples that contain very low fractions of dd-cfDNA are less informative regarding the donor genotype, making the inference harder. The two-genome method performs slightly better in differentiating between grades of acute rejection, although the differences were mostly non-significant; therefore, its dd-cfDNA estimates may be slightly more accurate (FIG. 2D). We conclude that donor genotyping is not required for lung transplant recipients. Donor genotyping can also be avoided in heart transplant recipients, but the accuracy of the test may be reduced slightly.

Quantifying Donor-Derived cfDNA in Bone Marrow Transplant Recipients

Because bone marrow donors are often close relatives of the recipients, the assumption that the donor is randomly selected from the population no longer holds. Chromosomes of closely-related individuals contain long segments of identical genotype. These segments are said to be identical by descent (IBD). The abundance and length of the IBD segments depend on the number of meioses separating the two chromosomes and the recombination rate. Ignoring IBD may lead to under-estimation of dd-cfDNA level. We therefore extended our model account for possible IBD by learning recipient-donor relatedness. We implemented a Hidden Markov Model (HMM) with three states (FIG. 1; see Methods Section infra for details): when there is no IBD (IBD=0), the model emission probabilities are similar to the above unrelated donor-recipient model; when one pair of chromosomes is IBD (IBD=1), the genotype of one donor allele will be similar to one of the recipient alleles and the other donor allele likelihood depends on its abundance in the population (independently of the recipient genotype); lastly, when both chromosome pairs are in IBD (IBD=2) the recipient and donor genotypes are identical. In our model, transitions between IBD states can occur only between pre-calculated 2 centimorgan blocks. Transition probabilities depend on the recipient-donor relatedness, which is represented by the number of meioses separating each pair of donor-recipient chromosomes (FIG. 1B indicated by (b)). In other words, in our model, the donor genotype depends on the population allele frequency and the recipient genotype according to the local IBD state.

### Accuracy of Dd-cfDNA Level Estimations in Bone Marrow Transplant Recipients

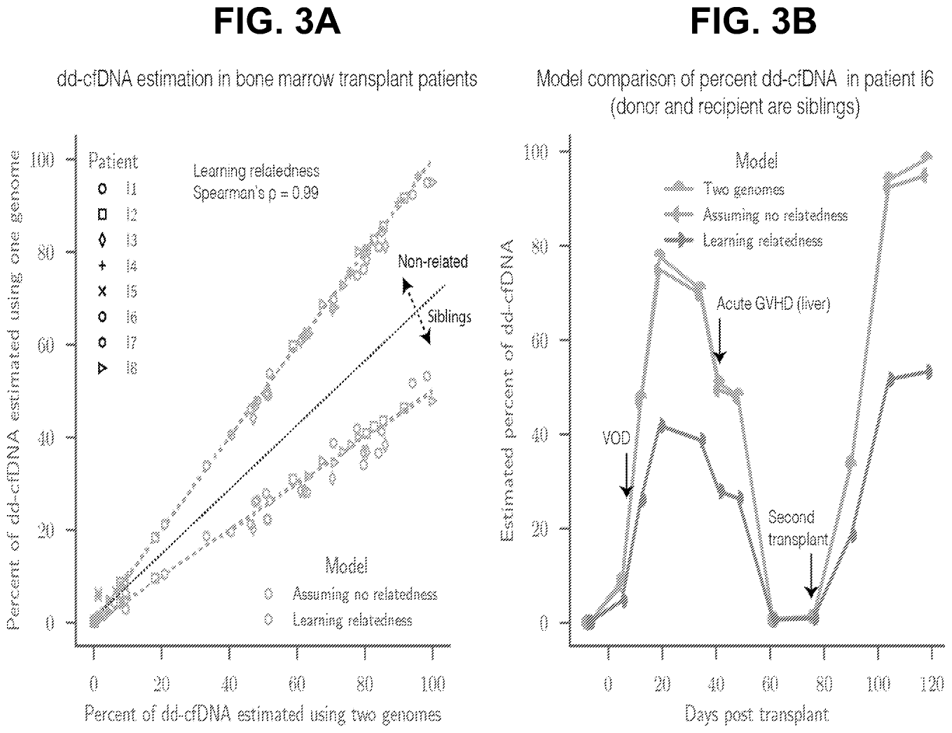

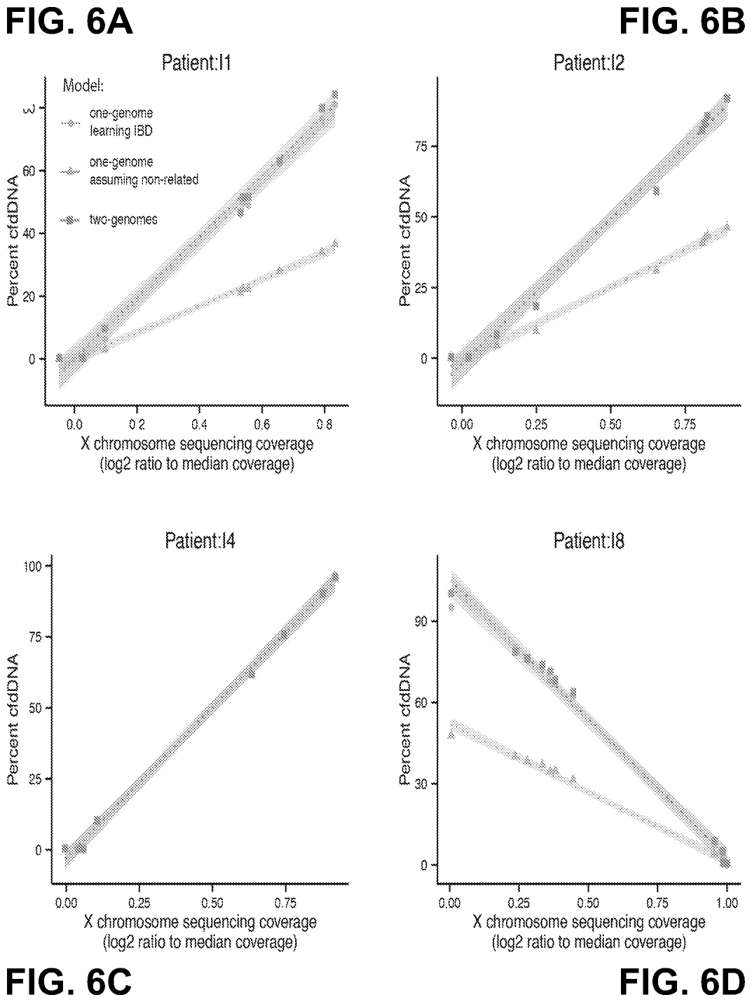

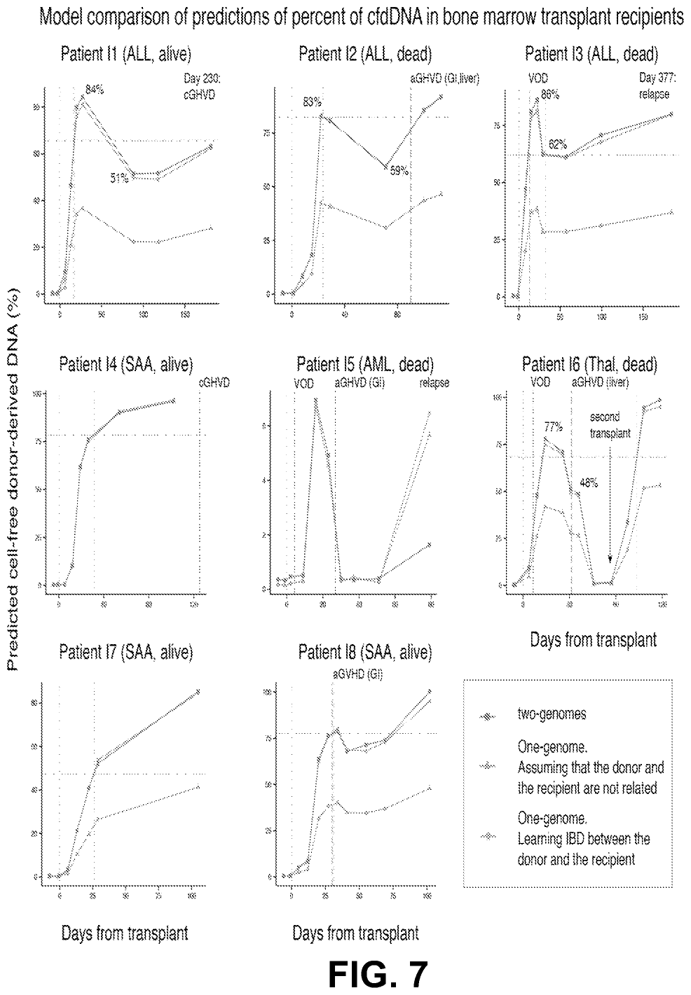

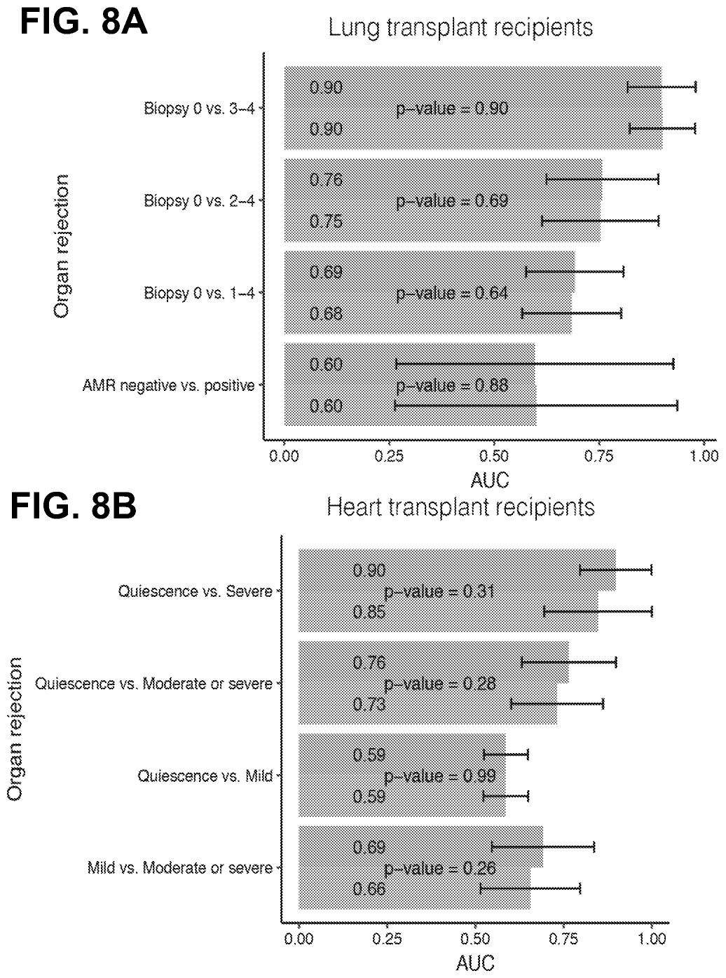

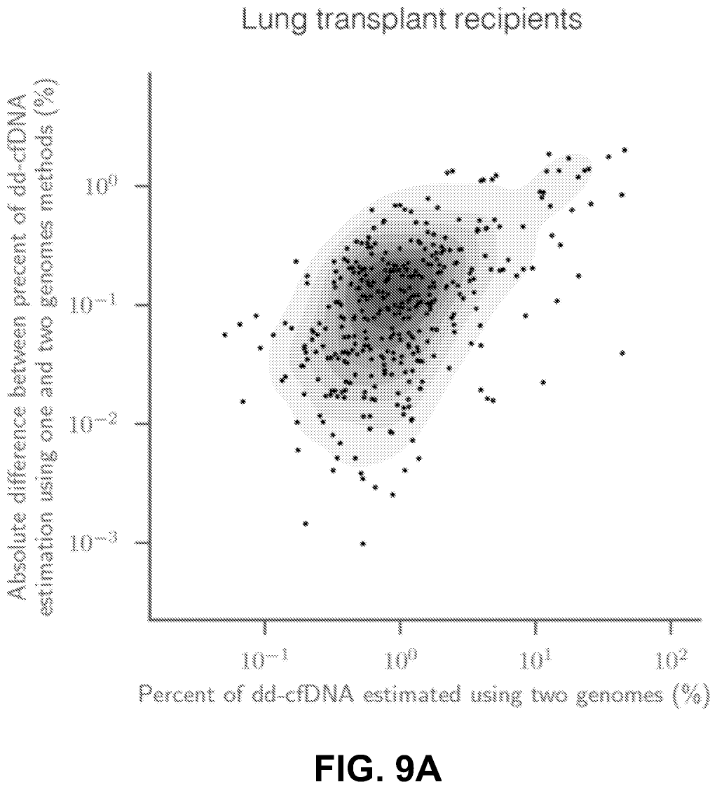

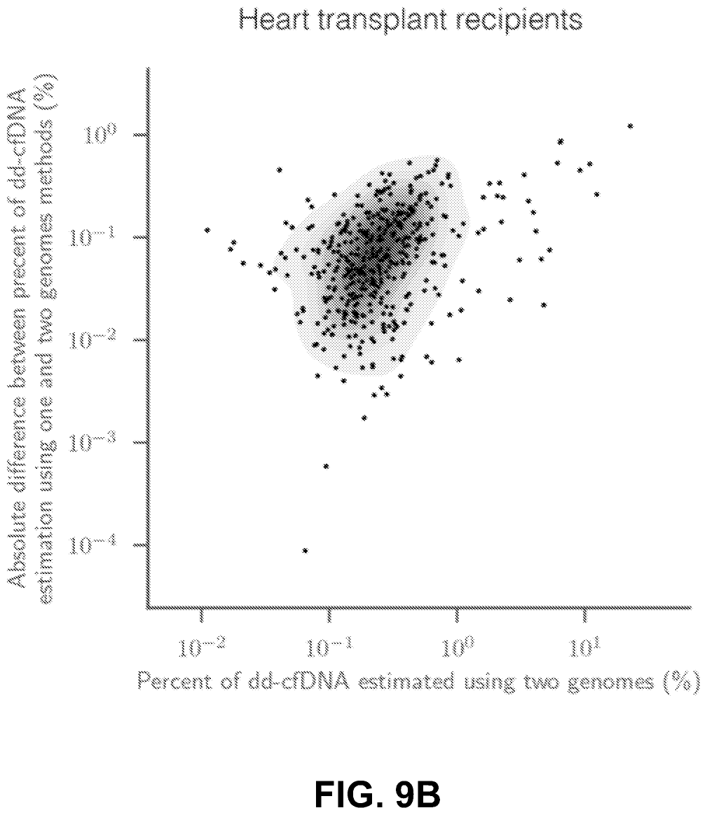

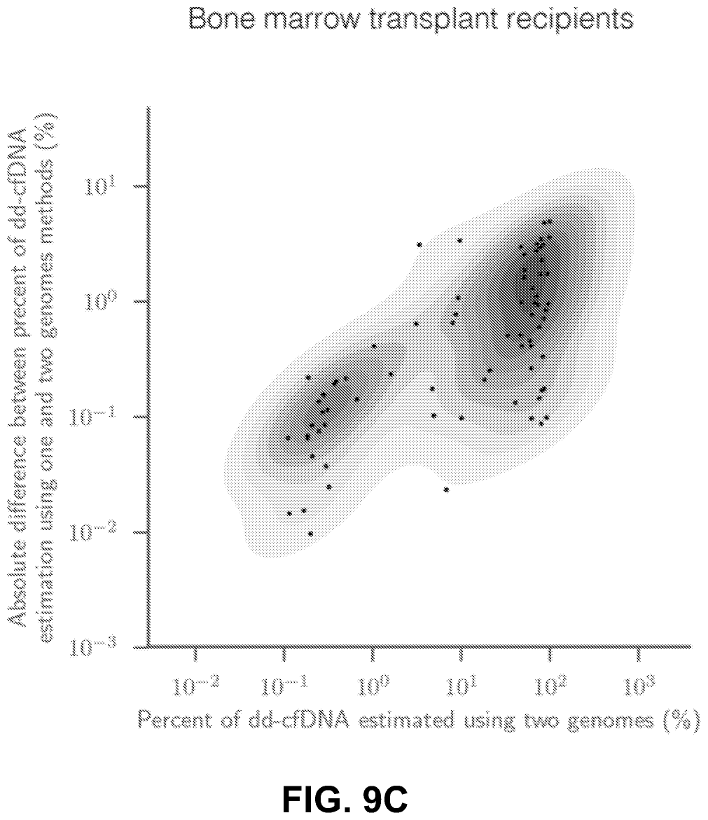

To evaluate the performance of the refined one-genome method, we applied it to 76 samples from 8 bone marrow transplant recipient patients (FIGS. 3A-B, FIG. 5). Two of the donors (for patients I4 and I5) were unrelated to the recipients and six were siblings of the recipients. As expected, the naïve implementation of the one-genome method underestimates dd-cfDNA in sibling donors (FIGS. 3A-B) that share about 50% of their genotype due to IBD (FIGS. 3A-B), but not in unrelated donors. When our model is set to learn the relationship between the donor and the recipient, its dd-cfDNA level estimates match the two-genomes method (Pearson's R²=1, Spearman's ρ=0.99; FIG. 3A). Reassuringly, these predictions strongly correlate with the fraction of reads originating from the X chromosomes when the donor and recipient sex is different (FIGS. 6A-D). We conclude that accurate estimation of dd-cfDNA in bone marrow recipients does not require donor genotyping. These results may also apply to other settings, such as kidney transplants.

### Differentiating Between Relapse and Graft Versus Host Disease in Bone Marrow Transplant Recipients

The success of bone marrow transplants is often impaired by cancer relapses and graft versus host disease (GVHD). Diagnosing and differentiating between the two remains a major challenge in the field. The current gold standard for a successful engraftment is absolute neutrophil count greater than 500 for three consecutive days. This corresponds to 47-82% dd-cfDNA in our patients (FIGS. 6A-D). We notice that in patients who relapse (patients I3) or have acute GVHD (patients I1 and I8) or chronic GVHD (patients I2), the level of cfDNA drops after reaching its peak (24%, 33%, 11% and 24%, respectively). Although our cohort is too small to assess significance, this observation suggests that GTD can be used to monitor bone marrow transplant health.

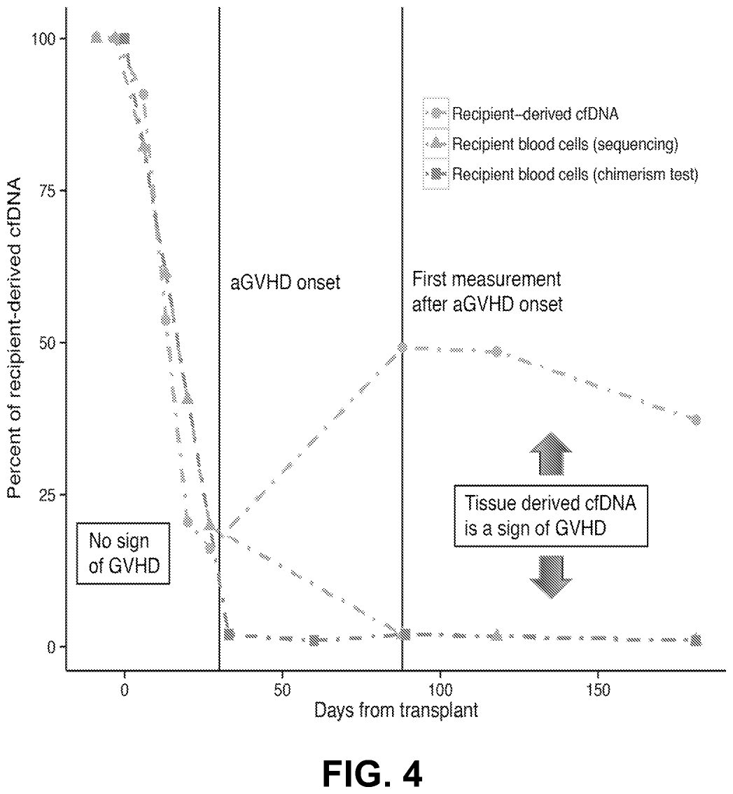

What are potential explanations for an increase in the level of cfDNA from recipient origin? In the case of a cancer relapse, the fraction of lymphocytes of recipient origin increases. The cfDNA will therefore reflect increasing levels of recipient-origin lymphocytes. On the other hand, in the case of GVHD, the fraction of lymphocytes from recipient origin does not increase. In this case, the increase in dd-cfDNA is caused by injury to recipient tissues. We therefore hypothesized that differences in the recipient-origin DNA in the cellular and plasma (cell-free) fractions can distinguish between relapse and GVHD. As a proof of principle, we sequenced both the cfDNA and the cellular fraction in patient I8. In agreement with our hypothesis, the two values match until the onset of the acute GVHD (since most cfDNA originates from lymphocytes) and then diverge—after the onset of GVHD, the cellular fraction remains low and cfDNA level increases (FIG. 4). This “N of one” experiment demonstrates the great potential of GTD to distinguish between relapses and GVHD—an urgent unmet need in the field.

### Methods Section

cfDNA Sequencing and Genotype Data Collection

The cfDNA sequencing and genotyping data for heart and lung transplant recipients was available from our previous studies [8,9]. Additional dd-cfDNA measurements were performed for bone marrow transplant patients (8 patients, 76 samples), using methods previously described [8,9]. In short, recipient plasma was collected at several time points before the transplant procedure (two time points) and at several time points after transplantation sequenced. cfDNA was purified from plasma and sequenced (Illumina HiSeq 200 or HiSeq 2500 1×50 bp or 2×100 bp). Donor and recipient genotyping was performed using Illumina whole-genome arrays HumanOmni2.5-8 or HumanOmni1 prior to the transplant.

Estimating Allele Representation in cfDNA Fragments

Several steps were applied to the cfDNA sequencing reads to achieve counts of allele representation for each genotyped SNP. First, low quality reads were filtered out (reads in which more than 50% of the base qualities are below 20). Second, reads were mapped to the human genome (UCSC version hg19) using bowtie2 [28] (with the following parameters: -D 20 -R 3 -N 0 -L 20 -i S,1,0.50 -I 20 -X 500 --no-mixed --no-discordant --no-unal -t) and SAMtools [29] was used to filter paired ends reads where one of the reads was unmapped (flags -f 3 -F 3852 for pair ends reads and -F 3844 for single end reads) or reads with P>0.05 to be mapped non-uniquely. Third, WASP [14] was applied to remove reads in which the mapping may be biased by the genotype. Fourth, duplicated reads (reads that map to the same exact location) were removed by scripts that selects randomly which of the duplicated reads to keep and is therefore not biased towards a specific genotype. Fifth, chromosomal coverage was computed using HTSeq [30]. Sixth, the number of cfDNA reads that contain each SNP allele was computed using SAMtools mpileup function. These counts were used as input for the model.

Estimating cfDNA Donor-Derived in Recipient that is Unrelated to the Donor

As vital organs such as heart and lungs are donated post-mortem, donors are usually unrelated to recipients. Therefore, our model assumes that the donor was randomly selected from some ancestral population. This is clearly an assumption—donors may have a mixed ancestry and their MHC is often matched to the recipient MHC—nonetheless we find that we can achieve good performance by making this assumption (we note that modeling of mixed ancestry did not improved the predictions). Given the population from which the donor was drawn, the prior probability of observing each allele in the donor is exactly the allele frequency in the population (assuming Hardy-Weinberg equilibrium). Since the donor population is unknown, the optimization function iterates over 1000 Genomes project populations and super-population [16] and selects the population that maximize the likelihood. The goal of the model is to estimate the fraction of cfDNA that is donor-derived (dd-cfDNA) given the recipient measured genotype and the cfDNA reads (FIG. 1 indicated by (a)).

Formally, let N be the number of bi-allelic SNP that were genotyped in the recipient; A and B denote the two possible alleles for SNP where i∈{1, 2, . . . , N}; (Rᵢ_{₁}, Rᵢ_{₂}) be the recipient true genotype in SNPᵢ; (Rᵢ_{₁}*,Rᵢ_{₂}*) be the recipient observed (measured) genotype in SNPᵢ; (Dᵢ_{₁}, Dᵢ_{₂}) be the donor true genotype in SNPᵢ; fᵢ_{_{A}}^{pop}^{ₘ} be the frequency of allele A of SNPᵢ in population m;

$${C}_{{i}_{j ∈ \{ 1 , 2 , … , {K}_{i} \}}}$$

be the true SNPᵢ allele in a cfDNA fragment that contains it; and

$${C}_{{i}_{j ∈ \{ 1 , 2 , … , {K}_{i} \}}}^{*}$$

be the observed allele of SNPᵢ from a sequencing read of this fragment. The observed data (R*, C*) is therefore the recipient measured genotype at N SNPs and the observed allele of these SNPs in cfDNA sequencing reads.

Lets also define the following model parameters (θ): d∈[0,1] is the fraction cfDNA fragments that are donor-derived (dd-cfDNA); eₛ ∈[10⁻⁹,10⁻²] is the sequencing error rate; e_{g} ∈[10⁻⁹,10⁻⁴] is the genotyping error rate; and Popₘ ∈{1, . . . , M} is one of M ancestral population and super populations of 1000 genomes project from which the donor is randomly drawn. The model sequencing and genotyping error rates were bound to technically realistic range. The goal of our model is to estimate d−the fraction of dd-cfDNA.

In our model, of the dependency of the observed recipient genotype of SNPᵢ on the true genotype involves the genotyping error rate. So, for example:

$$P ( ( {R}_{{i}_{1}}^{*} , {R}_{{i}_{2}}^{*} ) = AA \vert ( {R}_{{i}_{1}} , {R}_{{i}_{2}} ) ; {e}_{g} ) = {( 1 - {e}_{g} )}^{2} 1 \{ {R}_{{i}_{1}} = A , {R}_{{i}_{2}} = A \} + 2 ( {e}_{g} ) ( 1 - {e}_{g} ) 1 \{ {R}_{{i}_{1}} = A , {R}_{{i}_{2}} = B \} + {( {e}_{g} )}^{2} 1 \{ {R}_{{i}_{1}} = B , {R}_{{i}_{2}} = B \}$$

Similarly, the dependency of the observed allele in a sequencing read that map to SNPᵢ on the true allele of SNPᵢ in the cfDNA fragment that was sequenced involves the sequencing error rate (this also capture PCR amplifications errors):

P(Cᵢ_{ⱼ}*=A|Cᵢ_{ⱼ};eₛ)=(1−eₛ)1{Cᵢ_{ⱼ}=B}+(eₛ)1{Cᵢ_{ⱼ}=A}

Following the assumption that the donor was randomly drawn from a population, the genotype of SNPᵢ depends on SNPᵢ alleles frequencies in the population and therefore on which ancestral population is used to achieve the SNPᵢ alleles frequencies estimates:

$$P ( ( {D}_{{i}_{1}} = {d}_{1} , {D}_{{i}_{2}} = {d}_{2} ; {pop}_{m} ) = {f}_{{i}_{{d}_{1}}}^{{pop}_{m}} * {f}_{{i}_{{d}_{2}}}^{{pop}_{m}} , \\ where {d}_{1} ∈ \{ A , B \} and \\ {d}_{2} ∈ \{ A , B \}$$

Lastly, the probability that a cfDNA that maps to SNPᵢ contains a specific allele of SNPᵢ depends of the true genotype of the recipient and the donor and involves the fraction of donor-derived cfDNA (d); for example:

$$P ( {C}_{{i}_{j}} = A \vert {R}_{{i}_{1}} , {R}_{{i}_{2}} ) , ( {D}_{{i}_{1}} , {D}_{{i}_{2}} ) ; d ) = d * ( \begin{matrix}1 * 1 \{ {D}_{{i}_{1}} = A , {D}_{{i}_{2}} = A \} + \\ 0.5 * 1 \{ {D}_{{i}_{1}} = A , {D}_{{i}_{2}} = B \} + 0.5 * 1 \{ {D}_{{i}_{1}} = B , {D}_{{i}_{2}} = A \} + \\ 0 * 1 \{ {D}_{{i}_{1}} = B , {D}_{{i}_{2}} = B \}\end{matrix} ) + ( 1 - d ) * ( \begin{matrix}1 * 1 \{ {R}_{{i}_{1}} = A , {R}_{{1}_{2}} = A \} + \\ 0.5 * 1 \{ {R}_{{i}_{1}} = A , {R}_{{i}_{2}} = B \} + 0.5 * 1 \{ {R}_{{i}_{1}} = B , {R}_{{i}_{2}} = A \} + \\ 0 * 1 \{ {R}_{{i}_{1}} = B , {R}_{{i}_{2}} = B \}\end{matrix} )$$

Putting it together the likelihood of observing the recipient genotype and the sequencing reads that map to SNPᵢ is:

$${P}_{i} ( ( {R}_{{i}_{1}}^{*} , {R}_{{i}_{2}}^{*} ) , {C}_{{i}_{1}}^{*} , … {C}_{{i}_{{K}_{i}}}^{*} ; d , {e}_{s} , {e}_{g} , {pop}_{m} ) = {\prod }_{j = 1}^{{K}_{i}} {\sum }_{c , {r}_{1} , {r}_{1} , {d}_{1} , {d}_{2 , c} ∈ \{ A , B \}} ( \begin{matrix}P ( {C}_{{i}_{j}}^{*} \vert {C}_{{i}_{j}} = c ; {s}_{e} ) * \\ P ( {C}_{{i}_{j}} \vert ( {R}_{{i}_{1}} , {R}_{{i}_{2}} ) , ( {D}_{{i}_{1}} , {D}_{{i}_{2}} ) ; d ) * \\ P ( {D}_{{i}_{1}} = {d}_{1} , {D}_{{i}_{2}} = {d}_{2} ; {pop}_{m} ) * \\ P ( {R}_{{i}_{1}} = {r}_{1} , {R}_{{i}_{2}} = {r}_{2} ) * \\ P ( {R}_{{i}_{1}}^{*} , {R}_{{i}_{2}}^{*} \vert {R}_{{i}_{1}} = {r}_{1} , {R}_{{i}_{2}} = {r}_{2} ; {g}_{e} )\end{matrix} )$$

Although it is possible to model the probability of the recipient genotype (P(Rᵢ_{₁},Rᵢ_{₂}))

using population allele frequency data, we assume here a uniform probability since, in practice the genotyping error is very low and therefore the measured recipient genotype is highly informative on the true recipient genotype.

Finally, assuming that SNPs are independent (this is reasonable assumption because we used only genotyped SNPs), the likelihood function is:

$$ℒ ( {R}^{*} , {C}^{*} \vert θ ) = {\sum }_{i = 1}^{N} P ( ( {R}_{{i}_{1}}^{*} , {R}_{{i}_{2}}^{*} ) , {C}_{{i}_{1}}^{*} , … {C}_{{i}_{{K}_{i}}}^{*} \vert d , {s}_{e} , {g}_{e} , {pop}_{m} )$$

where R*, C* are genome-wide measured recipient genotype and all mapped sequencing reads correspondingly.

We use L-BFGS-B to minimize the negative log likelihood for each possible donor ancestral population and select the population that obtains the minimal negative log likelihood.

Estimating Donor-Derived cfDNA in Related Recipient and Donor

In contrast to lung and heart, bone marrow and other organs such as kidney, are often donated by individuals that are closely related to the recipient. Therefore, the assumption that the donor is drawn randomly from the population is no longer valid. Closely-related individuals share stretches of identical haplotypes that were inherited from a recent common ancestor, a phenomenon known as Identity By Descent (IBD). For each pair of chromosomes, IBD segments' length distribution and total length depend on the number of meioses from their Most Recent Common Ancestor (MRCA). The model accounts for IBD using a non-homogenous Hidden Markov Model (HMM) in which each position in the genome can be in one of three states IBD=0, IBD=1 or IBD=2 in which 0,1, or 2 pairs of chromosomes are identical by descent (FIG. 1 indicated by “b”). For efficiency and to avoid strong effects of linkage disequilibrium (LD), transitions are allowed only between ˜2 cM blocks, which are pre-calculated using a recombination rate map [22]. In each block, each one of the two haploid pairs of donor-recipient genomes can be in IBD or no-IBD state. The transitions between the IBD states for each haploid pair depend on the average genetic distance between the blocks and the marginal probability of the pair to be IBD, similar to the plink method [13]. In short, considering two haploids (c₁ and c₂) that share a common diploid ancestor with c₁ and c₂ separated by m 2 meiosis events, m=1−log₂(P_{IBD})) where P_{IBD} ∈[0,1] is the marginal probability of the pair to be in IBD state. We define l_{b,b+1} to be the genetic distance between two neighboring loci b,b+1 (here, approximated by the average genetic distance between blocks in cMorgan units). The probability of an odd number of recombination events

$${θ}_{b , b + 1} = \frac{1 - e \frac{- 2 * {l}_{b , b + 1}}{100}}{2} .$$

We also define y₁(θ_{b,b+1},m)=(1−θ_{b,b+1})^{m-2} and y₂(θ_{b,b+1})=(1−θ_{b,b+1})²+θ_{b,b+1}². The transition matrix for two haploids is:

$${T}_{b , b + 1} ( {m}_{i} ) = [ \begin{matrix}1 - ( \frac{1 - {y}_{1} ( {θ}_{b , b + 1} , {m}_{1} ) * {y}_{2} ( {θ}_{b , b + 1} )}{{2}^{{m}_{i} - 1} - 1} ) & \frac{1 - {y}_{1} ( {θ}_{b , b + 1} , {m}_{1} ) * {y}_{2} ( {θ}_{b , b + 1} )}{{2}^{{m}_{i} - 1} - 1} \\ 1 - {y}_{1} ( {θ}_{b , b + 1} , {m}_{1} ) * {y}_{2} ( {θ}_{b , b + 1} ) & {y}_{1} ( {θ}_{b , b + 1} , {m}_{1} ) * {y}_{2} ( {θ}_{b , b + 1} )\end{matrix} ]$$

where i∈{1,2}. The transition matrix for the IBD states of the two pairs of haploids is a simple combination of the two haploid pairs transition matrices and depends on their two IBD parameters: P_{IBD}^{I} and P_{IBD}^{II}. Similar to PLINK, we limit P_{IBD}^{I} and P_{IBD}^{II} to be at most 0.5. This excludes parent-child relations from the donor-recipient relationships. Although we did not address it in this work, dd-cfDNA of parent-child donor-recipient can be estimated by assuming that they are unrelated and accounting for the them sharing exactly 50% of their autosomal DNA by IBD (assuming that the parents are non-related).

The emissions probabilities of each SNP in each IBD state are similar to the likelihood function above with one difference—the probability of the donor genotype depends also on the recipient genotype (in addition to its dependence on the ancestral population):

$${P}_{i} ( ( {R}_{{i}_{1}}^{*} , {R}_{{i}_{2}}^{*} ) , {C}_{{i}_{1}}^{*} , … {C}_{{i}_{{K}_{i}}}^{*} \vert d , {e}_{s} , {e}_{g} , {pop}_{m} ) = { \prod }_{j = 1}^{{K}_{i}} {\sum }_{c , {r}_{1} , {r}_{1} , {d}_{1} , {d}_{2 , c} ∈ \{ A , B \}} ( \begin{matrix}P ( {C}_{{i}_{j}}^{*} \vert {C}_{{i}_{j}} = c ; {s}_{e} ) * \\ P ( {C}_{{i}_{j}} \vert ( {R}_{{i}_{1}} , {R}_{{i}_{2}} ) , ( {D}_{{i}_{1}} , {D}_{{i}_{2}} ) ; d ) * \\ P ( {D}_{{i}_{1}} = {d}_{1} , {D}_{{i}_{2}} = {d}_{2} \vert {R}_{{i}_{1}} = {r}_{1} , {R}_{{i}_{2}} = {r}_{2} , {IBD}_{i} ; {pop}_{m} ) * \\ P ( {R}_{{i}_{1}} = {r}_{1} , {R}_{{i}_{2}} = {r}_{2} ) * \\ P ( {R}_{{i}_{1}}^{*} , {R}_{{i}_{2}}^{*} \vert {R}_{{i}_{1}} = {r}_{1} , {R}_{{i}_{2}} = {r}_{2} ; {g}_{e} )\end{matrix} )$$

The following tables show P(Dᵢ_{₁}=d₁, Dᵢ_{₂}=d₂|Rᵢ_{₁}=r₁, Rᵢ_{₂}=r₂, IBD; popₘ) for a bi-allelic SNPᵢ, which has two possible alleles: A and B that are occur with frequency f_{A} and f_{B} in Popₘ respectively, for IBDᵢ=0,1,2.

Conditioning on IBDᵢ=0:

Conditioning on donor genotype

| | | AA | AB or BA | BB |
|---|---|---|---|---|
| Conditioning on the | AA | f_{A}² | 2f_{A}f_{B} | f_{B}² |
| recipient genotype | AB or BA | f_{A}² | 2f_{A}f_{B} | f_{B}² |
| | BB | f_{A}² | 2f_{A}f_{B} | f_{B}² |

Conditioning on IBDᵢ=1

Conditioning on donor genotype

| | | AA | AB or BA | BB |
|---|---|---|---|---|
| Conditioning on the | AA | f_{A} | f_{B} | 0 |
| recipient genotype | AB or BA | 0.5f_{A} | (0.5f_{A} + 0.5f_{B}) | 0.5f_{B} |
| | BB | 0 | f_{A} | f_{B} |

Conditioning on IBDᵢ=2

Conditioning on donor genotype

| | | AA | AB or BA | BB |
|---|---|---|---|---|
| Conditioning on the | AA | 1 | 0 | 0 |
| recipient genotype | AB or BA | 0 | 1 | 0 |
| | BB | 0 | 0 | 1 |

Putting it together the parameters of the model are: d−the fraction of dd-cfDNA, eₛ sequencing error probability, e_{g} genotyping probability and P_{IBD}^{I} and P_{IBD}^{II} IBD probability for the two haploid pairs. We used the Viterbi algorithm to calculate the likelihood of most likely path for specific parameter values, and optimize the likelihood using L-BFGS-B.

### Comparing One-Genome and Two-Genomes Methods Predictability of Organ Rejection

To assess how well each method dd-cfDNA predictions can be used to discriminate between different levels of heart and lung rejection, we computed the area under the curve (AUC) of the receiver operating characteristic (ROC): the dd-cfDNA prediction of one lung donation were doubled to match the levels of two lungs donations and measurements previous to 14 and 60 days following heart and lung transplant correspondingly were removed from the analysis. A two-sided DeLong test [31] (Implemented in R pROC package [32]) was used to assess the significance of the difference between two corresponding ROC curves.

### Data and Materials Availability

The bone marrow cohort sequence data have been deposited in the Sequence Read Archive (temporary submission id: SUB2077093). Code is available on github https://github.com/eilon-s/cfDNAG1.

### REFERENCES

1. Benden C, Edwards L B, Kucheryavaya A Y, Christie J D, Dipchand A I, Dobbels F, et al. The Registry of the International Society for Heart and Lung Transplantation: Sixteenth Official Pediatric Lung and Heart-Lung Transplantation Report—2013; focus theme: age. J Heart Lung Transplant. 2013; 32: 989-97. doi:10.1016/j.healun.2013.08.008
2. Stehlik J, Edwards L B, Kucheryavaya A Y, Benden C, Christie J D, Dobbels F, et al. The Registry of the International Society for Heart and Lung Transplantation: Twenty-eighth Adult Heart Transplant Report—2011. J Heart Lung Transplant. 2011; 30: 1078-94. doi:10.1016/j.healun.2011.08.003
3. Bloom R D, Goldberg L R, Wang A Y, Faust T W, Kotloff R M. An overview of solid organ transplantation. Clin Chest Med. 2005; 26: 529-43, v. doi:10.1016/j.ccm.2005.06.002
4. Lodhi S A, Lamb K E, Meier-Kriesche H U. Solid organ allograft survival improvement in the United States: the long-term does not mirror the dramatic short-term success. Am J Transplant. 2011; 11: 1226-35. doi:10.1111/j.1600-6143.2011.03539.x
5. Yusen R D, Christie J D, Edwards L B, Kucheryavaya A Y, Benden C, Dipchand A I, et al. The Registry of the International Society for Heart and Lung Transplantation: Thirtieth Adult Lung and Heart-Lung Transplant Report—2013; focus theme: age. J Heart Lung Transplant. 2013; 32: 965-78. doi:10.1016/j.healun.2013.08.007
6. Arcasoy S M, Berry G, Marboe C C, Tazelaar H D, Zamora M R, Wolters H J, et al. Pathologic interpretation of transbronchial biopsy for acute rejection of lung allograft is highly variable. Am J Transplant. 2011; 11: 320-8. doi:10.1111/j.1600-6143.2010.03382.x
7. Lo Y M, Tein M S, Pang C C, Yeung C K, Tong K L, Hjelm N M. Presence of donor-specific DNA in plasma of kidney and liver-transplant recipients. Lancet (London, England). 1998; 351: 1329-30.
8. De Vlaminck I, Valantine H A, Snyder T M, Strehl C, Cohen G, Luikart H, et al. Circulating cell-free DNA enables noninvasive diagnosis of heart transplant rejection. Sci Transl Med. 2014; 6: 241ra77. doi:10.1126/scitranslmed.3007803
9. De Vlaminck I, Martin L, Kertesz M, Patel K, Kowarsky M, Strehl C, et al. Noninvasive monitoring of infection and rejection after lung transplantation. Proc Natl Acad Sci USA. 2015; 112: 13336-41. doi:10.1073/pnas.1517494112
10. Snyder T M, Khush K K, Valantine H A, Quake S R. Universal noninvasive detection of solid organ transplant rejection. Proc Natl Acad Sci USA. 2011; 108: 6229-34. doi:10.1073/pnas.1013924108
11. Sung A D, Chao N J. Concise review: acute graft-versus-host disease: immunobiology, prevention, and treatment. Stem Cells Transl Med. United States; 2013; 2: 25-32. doi:10.5966/sctm.2012-0115
12. Lui Y Y N, Chik K-W, Chiu R W K, Ho C-Y, Lam C W K, Lo Y M D. Predominant hematopoietic origin of cell-free DNA in plasma and serum after sex-mismatched bone marrow transplantation. Clin Chem. 2002; 48: 421-7.
13. Purcell S, Neale B, Todd-Brown K, Thomas L, Ferreira M A R, Bender D, et al. PLINK: a tool set for whole-genome association and population-based linkage analyses. Am J Hum Genet. 2007; 81: 559-75. doi:10.1086/519795
14. van de Geijn B, McVicker G, Gilad Y, Pritchard J K. WASP: allele-specific software for robust molecular quantitative trait locus discovery. Nat Methods. 2015; 12: 1061-3. doi:10.1038/nmeth.3582
15. Li H. A statistical framework for SNP calling, mutation discovery, association mapping and population genetical parameter estimation from sequencing data. Bioinformatics. 2011; 27: 2987-93. doi:10.1093/bioinformatics/btr509
16. 1000 Genomes Project Consortium, Auton A, Brooks L D, Durbin R M, Garrison E P, Kang H M, et al. A global reference for human genetic variation. Nature. 2015; 526: 68-74. doi:10.1038/nature15393
17. Nelder J A, Mead R. A Simplex Method for Function Minimization. Comput J. 1965; 7: 308-313. doi:10.1093/comjn1/7.4.308
18. Bercovici S, Meek C, Wexler Y, Geiger D. Estimating genome-wide IBD sharing from SNP data via an efficient hidden Markov model of LD with application to gene mapping. Bioinformatics. 2010; 26: i175-82. doi:10.1093/bioinformatics/btq204
19. Rodriguez J M, Bercovici S, Huang L, Frostig R, Batzoglou S. Parente2: a fast and accurate method for detecting identity by descent. Genome Res. 2015; 25: 280-9. doi:10.1101/gr.173641.114
20. Browning B L, Browning S R. A fast, powerful method for detecting identity by descent. Am J Hum Genet. 2011; 88: 173-82. doi:10.1016/j.ajhg.2011.01.010
21. Thompson E A. Identity by descent: variation in meiosis, across genomes, and in populations. Genetics. 2013; 194: 301-26. doi:10.1534/genetics.112.148825
22. Kong A, Thorleifsson G, Gudbjartsson D F, Masson G, Sigurdsson A, Jonasdottir A, et al. Fine-scale recombination rate differences between sexes, populations and individuals. Nature. 2010; 467: 1099-103. doi:10.1038/nature09525
23. Grskovic M, Hiller D J, Eubank L A, Sninsky J J, Christopherson C, Collins J P, et al. Validation of a Clinical-Grade Assay to Measure Donor-Derived Cell-Free DNA in Solid Organ Transplant Recipients. J Mol Diagn. 2016; 18: 890-902. doi:10.1016/j.jmoldx.2016.07.003
24. Beck J, Bierau S, Balzer S, Andag R, Kanzow P, Schmitz J, et al. Digital droplet PCR for rapid quantification of donor DNA in the circulation of transplant recipients as a potential universal biomarker of graft injury. Clin Chem. 2013; 59: 1732-41. doi:10.1373/clinchem.2013.210328
25. De Vlaminck I, Khush K K, Strehl C, Kohli B, Luikart H, Neff N F, et al. Temporal response of the human virome to immunosuppression and antiviral therapy. Cell. 2013; 155: 1178-87. doi:10.1016/j.cell.2013.10.034
26. Snyder M W, Kircher M, Hill A J, Daza R M, Shendure J. Cell-free DNA Comprises an In Vivo Nucleosome Footprint that Informs Its Tissues-Of-Origin. Cell. 2016; 164: 57-68. doi:10.1016/j.cell.2015.11.050
27. Jun G, Flickinger M, Hetrick K N, Romm J M, Doheny K F, Abecasis G R, et al. Detecting and estimating contamination of human DNA samples in sequencing and array-based genotype data. Am J Hum Genet. 2012; 91: 839-48. doi:10.1016/j.ajhg.2012.09.004
28. Langmead B, Salzberg S L. Fast gapped-read alignment with Bowtie 2. Nat Methods. 2012; 9: 357-9. doi:10.1038/nmeth.1923
29. Li H, Handsaker B, Wysoker A, Fennell T, Ruan J, Homer N, et al. The Sequence Alignment/Map format and SAMtools. Bioinformatics. 2009; 25: 2078-9. doi:10.1093/bioinformatics/btp352
30. Anders S, Pyl P T, Huber W. HTSeq—a Python framework to work with high-throughput sequencing data. Bioinformatics. 2015; 31: 166-9. doi:10.1093/bioinformatics/btu638
31. DeLong E R, DeLong D M, Clarke-Pearson D L. Comparing the areas under two or more correlated receiver operating characteristic curves: a nonparametric approach. Biometrics. 1988; 44: 837-45.
32. Robin X, Turck N, Hainard A, Tiberti N, Lisacek F, Sanchez J-C, et al. pROC: an open-source package for R and S+ to analyze and compare ROC curves. BMC Bioinformatics. 2011; 12: 77. doi:10.1186/1471-2105-12-77

What is claimed is:

1. A method of quantifying transplant-derived circulating donor-derived cell-free DNA in the absence of a donor genotype, comprising:
(a) extracting a blood sample from a transplant recipient;
(b) isolating circulating nucleotide acids from the plasma of the extracted blood sample;
(c) performing an unbiased sequencing on the isolated circulating nucleotide acids using a first computer-implemented method;
(d) estimating and quantifying the donor-derived cell-free DNA based on genotyping from only the transplant recipient using a second computer-implemented method; and
(e) outputting the quantified transplant-derived circulating donor-derived cell-free DNA,
wherein the method is performed in the absence of a donor genotype;
wherein both computer-implemented methods and the outputting are executed by a computer device, and
wherein the method does not use targeted PCR amplification.

2. The method as set forth in claim 1, wherein the genotyping from only the transplant recipient is based on genotyping array, cell-free DNA sequencing or low coverage whole genome sequencing followed by imputations.

3. The method as set forth in claim 1, wherein the transplant recipients are solid-organ transplant, bone marrow transplant recipients, or hematopoietic stem cell recipients.
